(19) United States
(12) Patent Application Publication
(10) Pub. No.: US 2021/0307595 A1
(43) Pub. Date: Oct. 7, 2021

(54) DISPOSABLE COVER FOR AVOIDING CONTAMINATION OF ENDOSCOPE

(71) Applicant: Nizam M. MEAH, Manvel, TX (US)

(72) Inventor: Nizam M. MEAH, Manvel, TX (US)

(21) Appl. No.: 16/842,562

(22) Filed: Apr. 7, 2020

(51) Int. Cl. A61B 1/00; A61B 50/00

(52) U.S. Cl. CPC A61B 1/00142; A61B 1/0014; A61B 2050/0088; A61B 2050/0071; A61B 50/00

(57) ABSTRACT

Disclosed is a disposable cover for avoiding contamination of an endoscope. The disposable cover includes an elongate hollow body configured to enclose at least a portion of the endoscope. The elongate hollow body includes a first opening configured at a first end portion of the elongate hollow body, a second opening configured at a second end portion of the elongate hollow body and an intermediate body portion extending between the first and second end portions. The disposable cover also includes at least one attachment means arranged on each of the first and second end portions.

### TECHNICAL FIELD

The present disclosure relates generally to hygiene for medical instruments, more particularly to, a disposable cover for avoiding contamination of an endoscope.

### BACKGROUND

Endoscopes are medical instruments generally used for visual inspection of internal body organs or tissues. The most common use of the endoscope is to visually inspect a digestive track. Typically, an endoscope includes parts like, a control body (or operating part), a light guide tube, an insertion tube, an endoscope connector and so forth. In use, the insertion tube is inserted into the digestive track to visually inspect the digestive track. Typically, after the use, the insertion tube is mainly washed and disinfected. The process of washing and disinfecting requires time and effort, which may affect the working efficiency of the endoscope.

Conventionally, disposable covers for the insertion tube are known in the art, which may be disposed after the use thereof, to address the above stated problem. However, parts like the control body or nearby parts of the control body, i.e. connecting parts of the light guide and insertion tubes are equally prone to contamination, because an endoscopist operates the endoscope by primarily using the said parts. Further, as stated above, the washing and disinfecting process is mainly carried out for the insertion tube, which may cause contamination of the control body or parts near to the control body. Although, the entire endoscope can be washed and disinfected to avoid such contamination, however, this may require considerable time and effort, which may adversely affect the working efficiency of the endoscope.

In light of the foregoing discussion, there exists a need to overcome the aforementioned drawbacks associated with the contamination of endoscope.

### SUMMARY

Various embodiments of the present disclosure provide a disposable cover for avoiding contamination of an endoscope.

In an embodiment, a disposable cover for avoiding contamination of an endoscope is disclosed. The disposable cover includes an elongate hollow body configured to enclose at least a portion of the endoscope. The elongate hollow body includes a first opening configured at a first end portion of the elongate hollow body, a second opening configured at a second end portion of the elongate hollow body, and an intermediate body portion extending between the first and second end portions. The disposable cover also includes at least one attachment means arranged on each of the first and second end portions.

In another embodiment, a disposable cover for avoiding contamination of an endoscope is disclosed, and the disposable cover includes an elongate hollow body configured to enclose at least a portion of the endoscope. The elongate hollow body includes a first opening configured at a first end portion of the elongate hollow body, a second opening configured at a second end portion of the elongate hollow body, and an intermediate body portion extending between the first and second end portions. The elongate hollow body is made of an elastic biocompatible material allowing the elongate hollow body to be removably arranged on at least the portion of the endoscope.

In yet another embodiment, a disposable cover for avoiding contamination of an endoscope is disclosed, and the disposable cover includes an elongate hollow body configured to enclose at least a portion of the endoscope. The elongate hollow body includes a first opening configured at a first end portion of the elongate hollow body, a second opening configured at a second end portion of the elongate hollow body, and an intermediate body portion extending between the first and second end portions. The disposable cover also includes a zipper arranged along a length of the elongate hollow body, wherein the zipper is operable to allow the elongate hollow body to enclose at least the portion of the endoscope.

Other aspects and example embodiments are provided in the drawings and the detailed description that follows.

### BRIEF DESCRIPTION OF THE FIGURES

For a more complete understanding of example embodiments of the present technology, reference is now made to the following descriptions taken in connection with the accompanying drawings in which:

FIGS. 1-4 are perspective views of disposable covers for avoiding contamination of an endoscope, in accordance with various embodiments of the present disclosure; and

FIG. 5 is a perspective view of the disposable cover of FIG. 1 arranged on the endoscope, in accordance with an embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification is not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

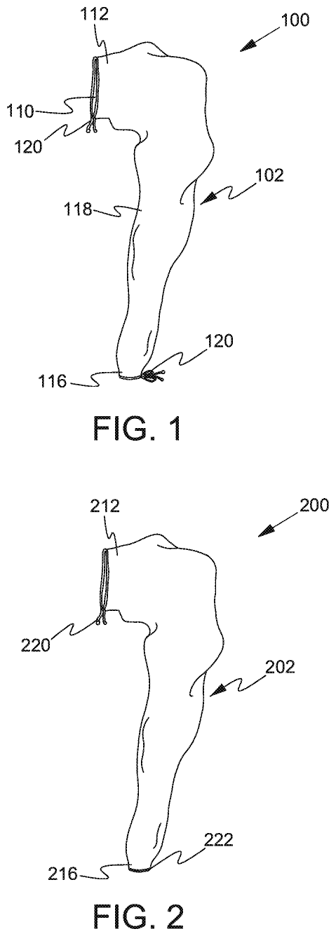

Referring now to the drawings, FIG. 1 is a perspective view of a disposable cover 100 for avoiding contamination of an endoscope (not shown), in accordance with an embodiment of the present disclosure. As shown, the disposable cover 100 includes an elongate hollow body 102 configured to enclose at least a portion of the endoscope, explained in greater detail herein later. The elongate hollow body 102 includes a first opening 110 configured at a first end portion 112 of the elongate hollow body 102. The elongate hollow body 102 also includes a second opening (not shown) configured at a second end portion 116 of the elongate hollow body 102. It will be appreciated that the second opening is similar to the first opening 110 and configured at the second end portion 116, opposite to the first end portion 112. The elongate hollow body 102 further includes an intermediate body portion 118 extending between the first and second end portions 112, 116.

In an example, the elongate hollow body 102 may be a hollow cylindrical structure. Typically, the elongate hollow body 102 is a hollow elongate structure having either a uniform or a non-uniform diameter (or structural dimensions). For example, the first and second end portions 112, 116 of the elongate hollow body 102 may be diametrically smaller as compared to a diameter of the intermediate body portion 118. Alternatively, the first and second end portions 112, 116 and the intermediate body portion 118 may include a same diameter.

In an exemplary embodiment, the elongate hollow body 102 includes a length from about 20 to about 40 centimeters. It will be appreciated that the length of the elongate hollow body is primarily constituted by a length of the intermediate body portion 118. Additionally, the elongate hollow body 102 may include a diameter from about 5 to about 15 centimeters.

In an embodiment, the elongate hollow body 102 is made of a biocompatible material. For example, the elongate hollow body 102 may be made of one of a biocompatible plastic, a biocompatible rubber, a biocompatible fabric or any combination thereof. Typically, the elongate hollow body 102 is a sheet or a layer of such biocompatible martial, which is foldable in nature. In another embodiment, the elongate hollow body 102 may be made of a non-biocompatible material, such as a sheet of non-biocompatible plastic or fabric.

The disposable cover 100 also includes at least one attachment means arranged on each of the first and second end portions 112, 116. According to an embodiment, the disposable cover 100 includes a similar attachment means arranged on each of the first and second end portions 112, 116. For example, the at least one attachment means includes a first attachment means 120 arranged on each of the first and second end portions 112, 116. In an embodiment, as shown in FIG. 1, the first attachment means 120 is a pull-string. It will be evident that, for the first attachment means 120 to be pull-string, each of the first and second end portions 112, 116 includes a closed loop for accommodating the pull-string therein. Further, the closed loop includes a slit from which both ends of the pull-string extend outside. In use, the both ends of the pull-string can be pulled out and a knot can be tied using the same for allowing the first and second end portions 112, 116 to be tied down to a portion of the endoscope, which is explained in greater detail herein later. In another embodiment, the first attachment means 120 may be an elastic band. In such instance, each of the first and second end portions 112, 116 also includes a closed loop for accommodating the elastic band therein.

Referring now to FIG. 2, illustrated is a perspective view of a disposable cover 200, in accordance with another embodiment of the present disclosure. Specifically, the disposable cover 200 includes different attachment means, i.e. the at least one attachment means includes a first attachment means arranged on a first end portion and a second attachment means arranged on a second end portion of an elongate hollow body. As shown, the disposable cover 200 includes an elongate hollow body 202 having first and second end portions 212, 216. Further, a first attachment means 220 is arranged on the first end portion 212 and a second attachment means 222 is arranged on the second end portion 216. As shown, the first attachment means 220 is a pull-string and the second attachment means 222 is an elastic band. However, it will be evident that, according to another embodiment, the first attachment means 220 may be an elastic band and the second attachment means 222 may be a pull-string.

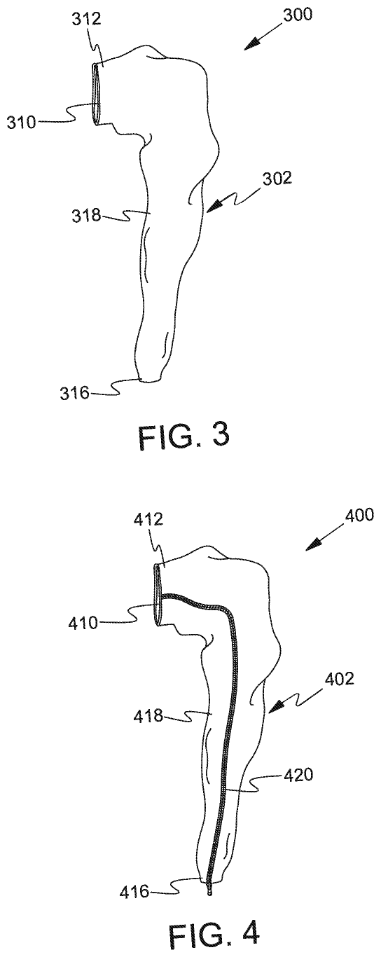

Referring now to FIG. 3, illustrated is a perspective view of a disposable cover 300, in accordance with yet another embodiment of the present disclosure. The disposable cover 300 is substantially, structurally and functionally similar to the disposable covers 100, 200 of FIGS. 1 and 2, respectively. For example, the disposable cover 300 also includes an elongate hollow body 302 having a first opening 310 configured at a first end portion 312 of the elongate hollow body 302, a second opening (similar to the first opening 310, not shown) configured at a second end portion 316 of the elongate hollow body 302, and an intermediate body portion 318 extending between the first and second end portions 312, 316. However, the disposable cover 300 lacks attachment means, such as the first attachment means 120 and the first and second attachment means 220, 222 of the disposable covers 100 and 200, respectively. Specifically, the elongate hollow body 302 is made of an elastic biocompatible material allowing the elongate hollow body 302 to be removably arranged on at least the portion of the endoscope (not shown), which is explained in greater detail herein later.

According to an embodiment, the elastic biocompatible material of the elongate hollow body 302 is a biocompatible rubber. Additionally, the elastic biocompatible material may include a combination of a biocompatible rubber with one of a biocompatible fabric or a biocompatible plastic. Alternatively, the elongate hollow body 102 may be made of a flexible non-biocompatible material. Typically, the elongate hollow body 302 is made of stretchable (of flexible) material (which may be biocompatible or non-biocompatible) that can be stretched for being snugly fitted over at least the portion of the endoscope.

According to an embodiment, the elongate hollow body 302 may include a length from about 20 to about 40 centimeters. Furthermore, the elongate hollow body 302 may include a diameter from about 5 to about 15 centimeters. Moreover, the elongate hollow body 302 may either include a uniform or a non-uniform diameter (or structural dimensions) along a length of thereof.

Referring now to FIG. 4, illustrated is a perspective view of a disposable cover 400, in accordance with still another embodiment of the present disclosure. The disposable cover 400 includes an elongate hollow body 402 having a first opening 410 configured at a first end portion 412 of the elongate hollow body 402, a second opening (similar to the first opening 410, not shown) configured at a second end portion 416 of the elongate hollow body 402, and an intermediate body portion 418 extending between the first and second end portions 412, 416. The disposable cover 400 also includes a zipper 420 arranged along a length of the elongate hollow body 402. The zipper 420 is operable to allow the elongate hollow body 402 to enclose at least a portion of the endoscope, which is explained in greater detail herein later.

According to an embodiment, the elongate hollow body 402 is made of one of a biocompatible plastic, a biocompatible rubber, a biocompatible fabric or any combination thereof. Alternatively, the elongate hollow body 402 may be made of a non-biocompatible material, such as non-biocompatible plastic or rubber. Further, the elongate hollow body 402 may include a length from about 20 to about 40 centimeters. Moreover, the elongate hollow body 402 may include a diameter from about 5 to about 15 centimeters. Additionally, the elongate hollow body 402 may either include a uniform or a non-uniform diameter (or structural dimensions) along a length of thereof.

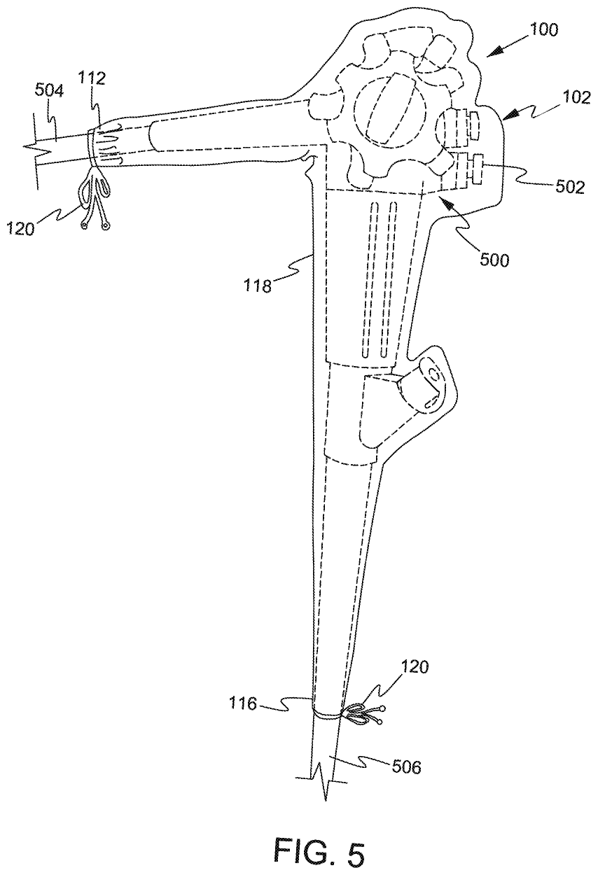

Referring now to FIG. 5, illustrated is a perspective view of the disposable cover 100 of FIG. 1 arranged on the endoscope 500 (shown with dotted lines), in accordance with an embodiment of the present disclosure. As shown, in use, the intermediate body portion 118 of the elongate hollow body 102 is configured to enclose a control body (or operating part) 502 of the endoscope 500. Further, the first and second end portions 112, 116 of the elongate hollow body 102 are fastened (or tied) to a portion of a light guiding tube 504 and an insertion tube 506, respectively, using the least one attachment means, i.e. the first attachment means 120 (which are pull-strings). Basically, while using the disposable cover 100 the elongate hollow body 102 is either received by the light guiding tube 504 or the insertion tube 506, through one of the first opening 110 or the second opening (not shown). Thereafter, once the entire disposable cover 100 is received by the endoscope 500, the elongate hollow body 102 is adjusted over the endoscope 500 in a manner such that the intermediate body portion 118 encloses the control body 502 and the first and second end portions 112, 116 rest on the light guiding tube 504 and the insertion tube 506 (respectively, or inversely). Finally, the first attachment means 120 (i.e. the pull-strings) are pulled and tied to the light guiding and insertion tubes 504, 506 for allowing the disposable cover 100 to be secured to the endoscope 500. This encloses at least the portion, such as the control body 502 and nearby portions of the control body 502, i.e. a portion of the light guiding and insertion tubes 504, 506. The endoscope 500 can be used in a diagnostic procedure with the disposable cover 100 arranged thereon, enclosing the control body 502 and nearby portions of the control body 502 to avoid contamination thereof. After use, the first attachment means 120 (i.e. the pull-strings) can be untied for removing the disposable cover 100 from the endoscope 500 for being disposed.

In will be appreciated that, while using the disposable cover 300 of FIG. 3 (which lacks attachment means, such as the pull-strings or elastic bands), the intermediate body portion 318, the first end portion 312 and the second end portion 316 of the elongate hollow body 302 are elastically secured to a control body (such as the control body 502, shown in FIG. 5), a light guiding tube (such as the light guiding tube 504) and an insertion tube (such as the insertion tube 506), respectively. Typically, in use, the entire disposable cover 300 is received by the endoscope 500 through one of the first opening 310 or the second opening. Thereafter, the elongate hollow body 302 is adjusted over the endoscope in a manner such that the intermediate body portion 318 encloses and secures to the control body and the first and second end portions 312, 316 rest and secure to the light guiding tube and the insertion tube (respectively or inversely).

In will be appreciated that, while using the disposable cover 300 of FIG. 3 (which lacks attachment means, such as the pull-string or elastic band), the intermediate body portion 318, the first end portion 312 and the second end portion 316 of the elongate hollow body 302 are elastically secured to a control body (such as the control body 502, shown in FIG. 5), a light guiding tube (such as the light guiding tube 504) and an insertion tube (such as the insertion tube 506), respectively. Typically, in use, the entire disposable cover 300 is received by the endoscope through one of the first opening 310 or the second opening. Thereafter, the elongate hollow body 302 is stretched and adjusted over the endoscope in a manner such that the intermediate body portion 318 encloses and secures to the control body and the first and second end portions 312, 316 rest and secure to the light guiding tube and the insertion tube (respectively or inversely). Additionally, after use, the elongate hollow body 302 (due to its elastic nature) can be pulled to remove from the endoscope 500 for being disposed.

In will be further appreciated that, while using the disposable cover 400 of FIG. 4, the intermediate body portion 418 of the elongate hollow body 402 is configured to enclose a control body (such as the control body 502) of the endoscope (such as the endoscope 500, shown in FIG. 5), and the first and second end portions 412, 416 of the elongate hollow body 402 are arranged on a portion of a light guiding tube (such as the light guiding tube 504) and an insertion tube (such as the insertion tube 506), respectively, when the zipper is in a closed state. Typically, in use, the zipper 420 is completely or partially opened from the second end portion 416 allowing the entire disposable cover 400 to be received by the endoscope via one of the light guiding tube or the insertion tube. Thereafter, the elongate hollow body 402 is adjusted over the endoscope in a manner such that the intermediate body portion 418 encloses the control body and the first and second end portions 412, 416 rest on the light guiding tube and the insertion tube (respectively or inversely). Finally, the zipper 420 is closed for securing the disposable cover 400 over the endoscope to enclose the control body and a portion of the light guiding and insertion tubes. After use, the elongate hollow body 402 is removed from the endoscope when the zipper 420 is in the open state. Typically, after the use of the disposable cover 400, the zipper 420 is opened (completely or partially) for removing the disposable cover 400 from the endoscope for being disposed.

Embodiments of the present disclosure substantially eliminate or at least partially address the aforementioned problems in the background and provide a disposable cover for avoiding contamination of an endoscope. The disposable cover of the present disclosure enables in keeping the endoscope away from any possible contamination, which may occur when the endoscope is used for any diagnostic procedure. The disposable cover enables in primarily enclosing or covering the control body (or operating part) and some other parts of the endoscope while the endoscope is in use, and after the diagnostic procedure is over, the disposable cover is removed from the endoscope for disposal thereof. Further, the disposable cover of the present disclosure enables in reducing overall time and effort that is generally required for cleaning and disinfecting the endoscope after use. This may help in improving working efficiency of the endoscope. Additionally, the disposable cover of the present disclosure can be conveniently used in conjunction with any endoscope sheath or cover that is used as protective cover.

The embodiments illustrated and described herein as well as embodiments not specifically described herein but within the scope of the aspects of the invention constitute an exemplary disposable cover for avoiding contamination of an endoscope.

The benefits and advantages described above may relate to one embodiment or may relate to several embodiments. The embodiments are not limited to those that solve any or all of the stated problems or those that have any or all of the stated benefits and advantages.

Aspects of any of the examples described above may be combined with aspects of any of the other examples described to form further examples without losing the effect sought.

The above description is given by way of example only and various modifications may be made by those skilled in the art. The above specification, examples and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the spirit or scope of this specification.

What is claimed is:

1. A disposable cover for avoiding contamination of an endoscope, the disposable cover comprising:
an elongate hollow body configured to enclose at least a portion of the endoscope, the elongate hollow body comprising
a first opening configured at a first end portion of the elongate hollow body,
a second opening configured at a second end portion of the elongate hollow body,
an intermediate body portion extending between the first and second end portions, and
at least one attachment means arranged on each of the first and second end portions.

2. The disposable cover as claimed in claim 1, wherein the at least one attachment means comprises a first attachment means arranged on each of the first and second end portions.

3. The disposable cover as claimed in claim 2, wherein the first attachment means is an elastic band.

4. The disposable cover as claimed in claim 2, wherein the first attachment means is a pull-string.

5. The disposable cover as claimed in claim 1, wherein the at least one attachment means comprises a first attachment means arranged on the first end portion and a second attachment means arranged on the second end portion.

6. The disposable cover as claimed in claim 5, wherein the first attachment means is an elastic band and the second attachment means is a pull-string.

7. The disposable cover as claimed in claim 5, wherein the first attachment means is a pull-string and the second attachment means is an elastic band.

8. The disposable cover as claimed in claim 1, wherein the elongate hollow body is made of one of a biocompatible plastic, a biocompatible rubber, a biocompatible fabric or any combination thereof.

9. The disposable cover as claimed in claim 1, wherein the elongate hollow body includes a length from about 20 to about 40 centimeters.

10. The disposable cover as claimed in claim 1, wherein, in use, the intermediate body portion of the elongate hollow body is configured to enclose a control body of the endoscope, and the first and second end portions of the elongate hollow body are fastened to a portion of a light guiding tube and an insertion tube, respectively, using the least one attachment means.

11. A disposable cover for avoiding contamination of an endoscope, the disposable cover comprising:
an elongate hollow body configured to enclose at least a portion of the endoscope, the elongate hollow body comprising
a first opening configured at a first end portion of the elongate hollow body,
a second opening configured at a second end portion of the elongate hollow body, and
an intermediate body portion extending between the first and second end portions,
wherein the elongate hollow body is made of an elastic biocompatible material allowing the elongate hollow body to be removably arranged on at least the portion of the endoscope.

12. The disposable cover as claimed in claim 11, wherein the elastic biocompatible material comprises a biocompatible rubber.

13. The disposable cover as claimed in claim 11, wherein the elastic biocompatible material comprises a combination of a biocompatible rubber with one of a biocompatible fabric or a biocompatible plastic.

14. The disposable cover as claimed in claim 11, wherein the elongate hollow body includes a length from about 20 to about 40 centimeters.

15. The disposable cover as claimed in claim 11, wherein, in use, the intermediate body portion, the first end portion and the second end portion of the elongate hollow body are elastically secured to a control body, a light guiding tube and an insertion tube, respectively.

16. A disposable cover for avoiding contamination of an endoscope, the disposable cover comprising:
an elongate hollow body configured to enclose at least a portion of the endoscope, the elongate hollow comprising
a first opening configured at a first end portion of the elongate hollow body,
a second opening configured at a second end portion of the elongate hollow body,
an intermediate body portion extending between the first and second end portions, and
a zipper arranged along a length of the elongate hollow body, wherein the zipper is operable to allow the elongate hollow body to enclose at least the portion of the endoscope.

17. The disposable cover as claimed in claim 16, the elongate hollow body is made of one of a biocompatible plastic, a biocompatible rubber, a biocompatible fabric or any combination thereof.

18. The disposable cover as claimed in claim 16, wherein the elongate hollow body includes a length from about 20 to about 40 centimeters.

19. The disposable cover as claimed in claim 16, wherein, in use, the intermediate body portion of the elongate hollow body is configured to enclose a control body of the endoscope, and the first and second end portions of the elongate hollow body are arranged on a portion of a light guiding tube and an insertion tube, respectively, when the zipper is in a closed state.

20. The disposable cover as claimed in claim 16, wherein the elongate hollow body is operable to be removed from the endoscope when the zipper is in an open state.
